(19) United States
(12) Patent Application Publication
(10) Pub. No.: US 2016/0200649 A1
(43) Pub. Date: Jul. 14, 2016

(54) METHOD FOR PURIFYING ISOPROPYL ALCOHOL

(71) Applicant: LG CHEM, LTD., Seoul (KR)

(72) Inventors: Jong Suh PARK, Daejeon (KR); Sung Kyu LEE, Daejeon (KR); Joon Ho SHIN, Daejeon (KR); Jong Ku LEE, Daejeon (KR)

(73) Assignee: LG CHEM, LTD., Seoul (KR)

(21) Appl. No.: 14/912,545

(22) PCT Filed: Aug. 20, 2014
(86) PCT No.: PCT/KR2014/007738

§ 371 Date: Feb. 17, 2016

(30) Foreign Application Priority Data
Aug. 20, 2013 (KR) 10-2013-0098663
Aug. 20, 2013 (KR) 10-2013-0098699
Aug. 20, 2014 (KR) 10-2014-0108605

(51) Int. Cl. C07C 29/76; B01D 15/08

(52) U.S. Cl. CPC C07C 29/76; B01D 15/08

(57) ABSTRACT

Provided are a method of and a device for purifying isopropyl alcohol. Water may be effectively removed from a feed including water and isopropyl alcohol while consuming a minimum amount of energy, and therefore high-purity isopropyl alcohol may be obtained.

### TECHNICAL FIELD

The present invention relates to a method and a device for purifying IPA.

### BACKGROUND ART

Isopropyl alcohol (IPA) is used in various applications, for example, as a cleaning agent in the electronics industry to manufacture semiconductors or liquid crystal displays (LCDs).

IPA may be prepared using propylene or acetone. In most cases, in the process of preparing IPA, an IPA reaction product including a large amount of water is obtained, and the reaction product forms an azeotrope including water. That is, water having a boiling point of approximately 100° C. and IPA having a boiling point of 82.5° C. at a normal pressure form a common ratio of IPA of 87.9 wt % at a temperature of 80.4° C., and thus high-purity IPA should be efficiently prepared by removing water from the feed, and a large amount of energy is consumed to remove the water in a simple distillation process. As a method of obtaining high-purity IPA from the azeotrope, a distillation method of adding an azeotropic agent, which is a material for forming an extract or azeotrope, is known.

### DISCLOSURE

### Technical Problem

The present invention is directed to providing a method and a device for purifying IPA.

### Technical Solution

In one aspect, a method of purifying IPA is provided. An exemplary purification method includes, as shown in FIG. 1, removing water by providing a feed to a dehydration means (D) (hereinafter referred to as a “dehydration process”) and purifying the feed from which water is removed via a hydrogenation means (D) after introduction into a purification means (P) (hereinafter referred to as a “purification process”). According to the purification method of the present invention, in the process of purifying IPA using the dehydration means (D) and a divided wall column (DWC) 200, optimal operating conditions for the DWC to minimize a water content in an IPA product may be deduced, thereby purifying IPA to a high purity. In addition, IPA may be purified with high efficiency using one DWC, compared to when using a purification means (P) in which two general columns are connected.

Here, the term “removal of water” does not refer to 100% removal of water included in a feed, but refers to forming a rich flow having a high IPA content by providing the feed to a dehydration means (D), and removing water or performing a purification process. The term “rich flow” used herein may refer to a flow having a higher IPA content included in the flow passing through the hydrogenation means (D) or the purification means P than the content of IPA included in the feed before being provided to the dehydration means (D), and for example, a flow including IPA included in the flow passing through the dehydration means (D) or the purification means (P) at a content of 50 wt % or more, 80 wt % or more, 90 wt % or more, 95 wt % or more, or 99 wt % or more.

In one example, the feed provided to the dehydration means (D) in the dehydration process may include IPA and water. A water content of the feed, that is, a content of water in the feed, may be 5,000 ppm or less, for example, 3,000 ppm or less, 2,500 ppm or less or 2,200 ppm or less. In addition, the lower limit of the water content in the feed may be, for example, 1,200 ppm. The water content in the feed may serve as a very important factor for efficiency, and thus the water content of the feed is necessarily adjusted within the above range. A particular composition of the feed is not particularly limited as long as it includes IPA and water, and a water content is adjusted within the above range. Conventionally, depending on a method of preparing a feed including IPA, the feed may include various types of impurities, which may be efficiently removed by the above method.

In the method, the dehydration means (D) into which the feed is introduced may include columns 110 and 111 charged with an adsorbent. For example, once a feed having a water content of 3,000 ppm is introduced, the column 110 or 111 charged with the adsorbent may be installed to discharge the feed by decreasing the water content thereof to 500 ppm or less, for example, 400 ppm or less, or 300 ppm or less, through a dehydration process. Accordingly, in the method, the water content of the feed may be adjusted to 500 ppm or less, for example, 400 ppm or less or 300 ppm or less, by removing water from the feed provided from the dehydration means (D). Efficiency of a subsequent purification process may be increased by adjusting the water content to the above range using the columns 110 and 111.

In one example, as the adsorbent, any of a variety of adsorbents known in the art including a molecular sieve, a silica gel, activated alumina, activated carbon, and an ion exchange resin may be used, but the present invention is not limited thereto.

For example, as the molecular sieve of the dehydration means (D), a known molecular sieve may be used without particular limitation as long as it is installed to have the dehydration capability as described above. For example, as the molecular sieve, a zeolite-based molecular sieve, a silica-based molecular sieve, an alumina-based molecular sieve, a silica-alumina-based molecular sieve or a silicate-alumina-based molecular sieve may be used.

As the molecular sieve, for example, a molecular sieve having an average micropore size of approximately 1.0 to 5.0 Å or 2.0 to 4.0 Å may be used. In addition, a specific surface area of the molecular sieve may be, for example, approximately 100 to 1,500 m³/g. The dehydration capability of the dehydration means (D) may be suitably adjusted using the molecular sieve having the micropore size and specific surface area in the above ranges.

In one example, the dehydration means (D) may include, for example, at least two columns 110 and 111 as described above. FIG. 2 exemplarily shows a dehydration means including at least two columns 110 and 111 charged with a molecular sieve. As shown in FIG. 2, when at least two columns 110 and 111 are included in the dehydration means (D), and a method of alternately providing a feed to the plurality of columns 110 and 111 is employed, the process efficiency may further be increased.

The method may further include regenerating the molecular sieve by detaching water adsorbed to the molecular sieve during dehydration. The detachment process of the molecular sieve may be performed in the purification process after the dehydration process, and when the plurality of columns 110 and 111 are used, while the dehydration process is performed in one column 110, the detachment process of the molecular sieve may be formed in the other column 111.

The regeneration may be performed using argon, carbon dioxide or nitrogen, or a low alkane such as methane, ethane, propane or butane. In one example, the regeneration process may be performed using nitrogen gas. When nitrogen gas is used, the regeneration process may be performed at a temperature of approximately 175 to 320° C. or 180 to 310° C. In addition, an amount of the nitrogen gas provided for detachment may be adjusted to, for example, approximately 1,100 to 1,500 Nm³/hr. In the above range, the regeneration or detachment process may be effectively performed. However, the temperature and flow rate may be changed according to a specific type or amount of the molecular sieve used.

The exemplary dehydration means (D) may further include a membrane system, as well as the columns 110 and 111 charged with the adsorbent. For example, when the feed having a water content adjusted to 500 ppm or less is introduced through the above-described columns 110 and 111, the membrane system may be installed to discharge the feed in which the water content is adjusted to 500 to 1,200 ppm through the above-described membrane system 100 to 50 to 500 ppm, for example, 100 to 500 ppm or 150 to 500 ppm, through the second dehydration. When the water content is adjusted in the above range using the columns 110 and 111, efficiency of a subsequent purification process may be increased. The term “membrane system” used herein refers to a system or device for separating a fluid using a separation film.

As the membrane system, any system using a separation film, for example, a pervaporation system or a vapor permeation system, may be used without particular limitation.

The term “pervaporation” used herein means a method of providing a liquid feed to a pervaporation film and selectively permeating a material having an affinity to the film to increase a purity of the feed, and the material passing through the pervaporation film is discharged by evaporation in a constant vacuum state, and captured by being cooled in a cooler. The pervaporation system may be applied to the purification method of the present invention when the feed is a liquid. When the dehydration process is performed using the pervaporation system, before the DWC 200 is charged with the feed, water is selectively removed in the dehydration process, thereby economically yielding high-purity IPA, compared to when water is removed by a simple distillation process.

In one example, when the dehydration means (D) includes a pervaporation system, in the dehydration process, the introduction of the liquid feed into the pervaporation system during the dehydration process may be performed at a temperature of, for example, 40 to 120° C., 70 to 110° C. or 80 to 100° C., but the present invention is not particularly limited thereto. In addition, the introduction of the liquid feed into the pervaporation system may be performed under a pressure of, for example, 1.0 to 10.0 kg/cm², 2.0 to 8.0 kg/cm², 2.5 to 6.0 kg/cm², or 3.0 to 5.0 kg/cm². The dehydration process of the liquid feed may be effectively performed in the range of the above-described temperature and/or pressure. However, the range of the temperature and/or pressure may be suitably changed in consideration of a desired dehydration amount and the separation film used. For example, generally, as the temperature and the pressure are increased, permeability of the separation film may be increased, but the upper limits of the temperature and the pressure may be changed according to a type of the separation film and process conditions. In addition, as the temperature and the pressure are increased, a permeation rate and a permeation amount may be increased, but the upper limits may be adjusted within suitable ranges according to a type of the material for the separation film used and durability of the separation film.

The term “vapor permeation” refers to a film separation method for separating a desired gas through a separation film by evaporating a feed to bring the gas in contact with the separation film. In the purification method, when the feed is in a gaseous state, the vapor permeation may be preferably applied. When a dehydration process is performed using the vapor permeation system, an azeotropic point is not generated, and thus water may be more efficiently removed than when the dehydration process is performed by distillation, and therefore high-purity IPA may be economically obtained.

In one example, the vapor permeation system may be charged with the feed with which the vapor permeation system of the dehydration means (D) is charged at a temperature of a boiling point or more of a mixed composition of water and IPA. The introduction of a gas-phase feed into the vapor permeation system in the dehydration process may be performed at, for example, 90° C. or more, 100° C. or more, 110° C. or more, 120° C. or more or 150° C. or more, and the upper limit of the temperature at which the gas-phase feed is introduced may be changed according to thermal or chemical characteristics of the separation film used, and may be, but is not particularly limited to, for example, approximately 180° C. In addition, the introduction of the gas-phase feed into the vapor permeation system may be performed under a pressure of, for example, 1.0 to 10.0 kg/cm², 2.0 to 8.0 kg/cm² or 3.0 to 6.0 kg/cm². In the above-described temperature and/or pressure ranges, a process of dehydrating a gas-phase feed may be efficiently performed. However, the temperature and/or pressure ranges may be suitably changed in consideration of a desired dehydration amount and the type of the separation film used.

The separation film which can be used in the pervaporation system or vapor permeation system may be an organic separation film such as a polymer membrane, an inorganic separation film, or an organic/inorganic separation film manufactured by mixing an organic material and an inorganic material according to the type of material used, and for the dehydration means (D) of the present invention, various separation films known in the art may be used according to a desired separated component. For example, as the hydrophilic separation film, a separation film formed of a silica gel, a separation film formed of a polymer such as PVA or polyimide, or a zeolite separation film may be used, but these may be suitably changed in consideration of a desired dehydration amount and a composition of the feed. As the zeolite separation film, a zeolite film produced by Pervatech, a zeolite A separation film produced by i3nanotec, or a zeolite NaA separation film may be used, but the present invention is not limited thereto.

In addition, the pervaporation system or the vapor permeation system may include a vacuum device. The vacuum device is a device for forming a vacuum to allow a separable component of the feed in contact with the separation film to be easily separated from the film, and may be a device composed of a vacuum storage tank and a vacuum pump.

A purification process may be performed by providing the feed in which a water content is adjusted to 500 ppm or less through the dehydration process to a purification means (P). In one example, the purification means (P) may be a DWC.

Here, the DWC 200 is a device designed to distill a feed including three components, for example, having a low boiling point, a middle boiling point and a high boiling point. The DWC 200 is a device similar to a thermally coupled distillation column (Petlyuk column) in terms of a thermodynamic aspect. The thermally coupled distillation column has a structure in which a pre-separator and a main separator are thermally integrated. The column is designed to primarily separate low-boiling-point and high-boiling-point materials from the preliminary separator, and charge each of top and bottom parts of the pre-separator to a feed of the main separator and separate low-boiling-point, medium-boiling-point and high-boiling-point materials from the main separator. Accordingly, the DWC 200 is formed by installing a dividing wall 201 in the column and integrating a pre-separator into a main separator.

The DWC 200 may have the structure as shown in FIG. 3. FIG. 3 shows an exemplary DWC 200. As shown in FIG. 3, the exemplary column may have a structure which is divided by the dividing wall 210, and includes a condenser 202 disposed in an upper portion and a reboiler 203 in a lower portion. In addition, as virtually divided by a dotted line in FIG. 3, the DWC 200 may be divided into, for example, a top region 210 discharging a low-boiling-point flow, a bottom region 220 discharging a high-boiling-point flow, a feed inflow region 230 into which the feed is introduced, and a product outflow region 240 discharging a product. The feed inflow region 230 may include an upper inflow region 231 and a lower inflow region 232, and the product outflow region 240 may include an upper product outflow region 241 and a lower product outflow region 242. Here, the term “upper and lower inflow regions” may refer to upper and lower regions, respectively, created when a feed-providing part of a space divided by the dividing wall 201 in the structure of the DWC 200, that is, the feed inflow region 230, is divided into equal two parts in a length direction of the column. In addition, the term “upper and lower product outflow regions” may refer to upper and lower regions created when a space of a product releasing side, which is divided by the dividing wall 201 in the DWC 200, that is, the product outflow region 240, is divided into equal two parts in a length direction of the column. The term “low-boiling-point flow” refers to a flow in which relatively low-boiling-point components are rich among the feed flows including three components such as low-, middle- and high-boiling-point components, and the term “high-boiling point flow” refers to a flow in which relatively high-boiling-point components are rich among the feed flows including three of low-, medium- and high-boiling-point components.

In the purification method of the present invention, the feed with which the feed inflow region 230 of the DWC 200 is charged is purified in the DWC 200. In addition, the component having a relatively low boiling point in the feed introduced into the feed inflow region 230 is transferred to the top region 210, and the component having a relatively high boiling point is transferred to the bottom region 220. A component having a relatively low boiling point in the component transferred to the bottom region 220 is transferred to the product outflow region 240, and discharged as a product flow or transferred to the top region 210. However, a component having a relatively high boiling point in the component transferred to the bottom region 220 is discharged as a high-boiling-point flow. A part of the high-boiling-point flow discharged from the bottom region 220 is discharged as a high-boiling-point flow from the bottom region 220. A part of the high-boiling-point flow discharged from the bottom region 220 is discharged as a flow of the high-boiling-point component, and the rest is heated in the reboiler 203 and then reintroduced to the bottom region 220 of the DWC. Meanwhile, from the top region 210, a flow of the low-boiling-point component having a very rich water content may be discharged, the flow discharged from the top region 210 may be condensed in the condenser 202, a part of the condensed flow may be discharged, and the rest may be refluxed to the top region 210 of the DWC 200. In addition, the flow refluxed and discharged from the top region 210 is purified again in the DWC 200, thereby minimizing a content of IPA discharged from the top region 210 and maximizing a water content discharged from the top region 210.

A specific type of the DWC 200 that can be used in the purification method is not particularly limited. For example, the DWC having a general structure as shown in FIG. 3 is used, or a column modified in position or shape of the divided wall in the column in consideration of purification efficiency may also be used. In addition, a number of stages and an inner diameter of the column are not particularly limited either, and for example, the column may be designed based on the number of theoretical plates calculated from a distillation curve considering the composition of the feed.

In this method, the DWC 200 subjected to the purification process may be installed to discharge the feed having a water content adjusted to, for example, 500 ppm or less by reducing the water content in the feed to be 150 ppm or less, for example, 120 ppm or less, 110 ppm or less, 100 ppm or less, 80 ppm or less, 60 ppm or less, 50 ppm or less, 30 ppm or less or 10 ppm or less through the purification process. Accordingly, the purification process may include removing water from the feed provided to the DWC to adjust the water content of the feed to 150 ppm or less, for example, 120 ppm or less, 110 ppm or less, 100 ppm or less, 80 ppm or less, 60 ppm or less, 50 ppm or less, 30 ppm or less, or 10 ppm or less. According to the DWC 200, the water content may be adjusted to the above range, and IPA may be purified to a high purity at the same time.

The DWC 200 may be installed to provide, for example, the feed passing through the membrane system 100 to the feed inflow region 230 of the column. Accordingly, in the purification process, the feed in which the water content after the dehydration process is adjusted to 500 ppm or less may be provided to the feed inflow region 230 of the column. When the feed is provided to the DWC 200, in consideration of the composition of the feed, for example, as shown in FIG. 3, if the feed is provided to the upper inflow region 231, efficient purification can be performed.

Accordingly, the DWC 200 may be installed to discharge a product including purified IPA and having a water content of 150 ppm or less from a lower product outflow region 242, preferably, from a middle part of the lower product outflow region 242. That is, the purification method may include yielding the product including purified IPA and having a water content of 150 ppm or less from plates 50 to 90%, 55 to 80% or 60 to 75% of the number of theoretical plates calculated from the lower product outflow region 242, preferably, a top of the DWC 200. For example, when the number of theoretical plates of the DWC 200 is 100 plates, the product having a water content of 100 ppm or less may be discharged from 50 to 90 plates or 60 to 75 plates, and efficiency of the purification process may be further increased by adjusting a discharging location of the product as described above. The term “middle part of the lower product outflow region” used herein means a site at which the lower product outflow region 242 is divided into equal two parts in a length direction of the DWC 200.

The number of theoretical plates of the DWC 200 required to adjust a water content of a feed in which a water content is adjusted to 500 ppm or less as described above to be 150 ppm or less may be, but is not limited to, 70 to 120 plates, 80 to 110 plates or 85 to 100 plates, and may be suitably changed according to a flow amount of a charged feed and a process condition.

Meanwhile, due to a structural characteristic of the DWC 200 in which an internal circulation flow rate may be adjusted once a design is determined, unlike a Petlyuk column, flexibility according to the change in operating conditions is decreased, and precise copies of various disturbances and determination of an easily controllable control structure are required in an early stage of designing the column. Moreover, a designed column structure and operating conditions DWC 200 including a location of a supply plate, determination of sections of the dividing wall, a location of a plate for producing a middle-boiling-point material, the total number of theoretical plates, a distillation temperature and a distillation pressure are very limited, and a design structure including the number of plates of the column, and locations of a supply plate and a releasing plate, and operating conditions including a distillation temperature, a pressure, and a reflux ratio should be specially changed according to characteristics of a compound to be distilled. In the purification method of the present invention, as described above, an operating condition of the DWC 200 suitably designed to purify IPA may be provided to save energy and reduce a cost of equipment.

In one example, as described above, when the feed in which a water content is adjusted to 500 ppm or less is introduced into the DWC 200, and the water content in the feed is adjusted to 150 ppm or less in the DWC 200 through the purification process, the reflux ratio of the top region 210 of the DWC 200 may be adjusted in a range of 60 to 90, for example, 65 to 90, 70 to 85 or 75 to 85. For example, as the water content in the feed introduced into the DWC 200, it is necessary to considerably adjust the reflux ratio of the top region 210 for removing water in the feed and obtaining high-purity IPA, but in the purification method of the present invention, the water content in IPA obtained from the lower product outflow region 242 may be adjusted to be very low by adjusting the water content in the feed introduced into the DWC 200 to be 500 ppm or less, and adjusting the reflux ratio of the top region 210 in the DWC 200 within the specific range as described above.

The feed may be provided to the DWC 200 at a flow rate of, for example, approximately 5,000 to 13,000 kg/hr. In addition, a temperature of the provided feed may be adjusted to be, for example, approximately 50 to 135° C., 60 to 110° C. or 80 to 100° C. When the feed is provided at the above-described flow rate and temperature, suitable distillation efficiency may be achieved.

As described above, during the distillation performed by providing the feed in which a water content is adjusted to 500 ppm or less to the DWC 200, the operating temperature of the top region 210 of the DWC 200 may be adjusted to 40 to 120° C., for example, approximately 45 to 110° C. or 50 to 100° C. In this case, the operating pressure of the top region 210 of the DWC 200 may be adjusted to 0.1 to 10.0 kg/cm², for example, approximately 0.2 to 5.5 kg/cm², 0.3 to 4.5 kg/cm², 0.6 to 4.0 kg/cm² or 0.68 to 3.7 kg/cm². At such operating temperature and pressure, effective distillation according to the composition of the feed may be performed. In the specification, the pressure is, unless particularly defined otherwise, an absolute pressure.

The operating and pressure conditions in the DWC 200 may be changed according to the temperature and pressure conditions of the top region 210. In one example, when the temperature of the top region 210 of the DWC 200 is adjusted to 40 to 120° C., a temperature of release flow discharged from the lower product outflow region 242 of the DWC 200 may be adjusted to 60 to 130° C., for example, approximately 70 to 125° C., 75 to 120° C. or 77.3 to 120° C. In addition, when the pressure of the top region 210 of the DWC 200 is adjusted to 0.2 to 5.5 kg/cm², the operating pressure of the lower product outflow region 242 of the DWC 200 may be adjusted to 0.3 to 6.0 kg/cm², for example, approximately 0.5 to 5.0 kg/cm², 0.8 to 4.0 kg/cm² or 0.843 to 3.86 kg/cm². With such operating temperature and pressure, effective distillation according to a composition of the feed can be performed.

In addition, when the temperature of the top region 210 of the DWC 200 is adjusted to 40 to 120° C., the operating temperature of the top region 220 of the DWC 200 may be adjusted to 80 to 160° C., for example, approximately 90 to 160° C., 95 to 158° C., or 104 to 156° C. In addition, when the pressure of the top region 210 of the DWC 200 is adjusted to 0.2 to 5.5 kg/cm², the operating pressure of the bottom region 220 of the DWC 200 may be adjusted to 0.3 to 6.0 kg/cm², for example, approximately 0.8 to 5.0 kg/cm², 0.9 to 4.0 kg/cm², or 0.91 to 3.93 kg/cm². At such operating temperature and pressure, effective distillation according to the composition of the feed can be performed.

Here, the operating condition of the DWC 200 may be further adjusted when needed in consideration of purification efficiency.

Other conditions of the DWC 200 on which the purification process is performed, for example, the number of plates or inner diameter of each column, are not particularly limited. For example, the number of theoretical plates of the DWC 200 may be determined based on the number of theoretical plates calculated by a distillation curve of the feed. In addition, flow rates of the upper and lower discharged products from the DWC 200 may be set to achieve, for example, the above-described operating pressure and temperature.

In another aspect, a device for purifying IPA is provided. The exemplary purification device may be a device to be applied to the above-described purification method.

Accordingly, the purification device may include a dehydration means (D) installed to discharge the feed having a decreased water content of 500 ppm or less, for example, when the above-described feed is provided, and a purification means (P) in which a purification process is performed with respect to the feed passing through the dehydration means (D).

Specific descriptions related to the purification device may be the same as or similar to, for example, those described above.

The dehydration means (D) may be, for example, a column charged with an adsorbent.

In one example, as the adsorbent, various adsorbents known in the art may be used, and for example, a molecular sieve, a silica gel, activated alumina, activated carbon or an ion exchange resin, but the present invention is not limited thereto.

For example, as the molecular sieve of the dehydration means (D), a known molecular sieve may be used without particular limitation as long as it is installed to have the dehydration capability as described above. For example, as the molecular sieve, a zeolite-based molecular sieve, a silica-based molecular sieve, an alumina-based molecular sieve, a silica-alumina-based molecular sieve or a silicate-alumina-based molecular sieve may be used.

As the molecular sieve, for example, a molecular sieve having an average micropore size of approximately 1.0 to 5.0 Å or 2.0 to 4.0 Å may be used. In addition, a specific surface area of the molecular sieve may be, for example, approximately 100 to 1,500 m³/g. The dehydration capability of the dehydration means (D) may be suitably adjusted using the molecular sieve having the micropore size and specific surface area in the above ranges.

In one example, the dehydration means (D) may include a column charged with a molecular sieve. The dehydration means (D) may include, for example, at least two columns.

The exemplary dehydration means (D) may further include a membrane system, in addition to the columns.

As the membrane system, any system using a separation film, for example, a pervaporation system or a vapor permeation system, may be used without particular limitation.

As described above, the separation film which can be used in the pervaporation system or the vapor permeation system may be a separation film, an inorganic separation film, or an organic/inorganic separation film manufactured by mixing an organic material with an inorganic material for a polymer membrane according to a type of material used, and in the dehydration means (D) of the present invention, various separation films known in the art may be used in a variety of applications according to a desired separated component. For example, as a hydrophilic separation film, a separation film formed of a silica gel, a separation film formed of a polymer such as PVA or polyimide, or a zeolite separation film may be used, but these may be suitably changed in consideration of a desired dehydration rate and the composition of the feed. For example, as the zeolite separation film, a zeolite film manufactured by Pervatech, a zeolite A separation film manufactured by i3nanotec, or a zeolite NaA separation film may be used, but the present invention is not limited thereto. To maintain a strength of the separation film, a polymer separation film coated with an inorganic material may be used.

In addition, the pervaporation system or vapor permeation system may include a vacuum device. The vacuum device is a device for forming a vacuum to easily separate a component of the feed to be separated from a film after coming in contact with the separation film, for example, a device composed of a vacuum storage tank and a vacuum pump.

In one example, the purification device may include, for example, a purification means (P) into which the feed passing through the dehydration means (D) is introduced to perform a purification process.

The purification means (P) in which the purification process is performed may include, for example, at least one distillation column.

In one example, the purification means (P) may be a DWC.

Here, the DWC 200 may be installed such that, for example, the feed passing through the dehydration means (D) is provided in a feed inflow region 230, for example, an upper inflow region 231 of the DWC 200. In addition, the DWC 200 may be installed such that the product including IPA is discharged from the lower product outflow region 242, preferably, a middle part of the lower product outflow region 242.

Specific descriptions related to the DWC 200 are the same as those described in the above-described purification method, and thus will be omitted.

### Advantageous Effects

According to the present invention, high-purity IPA can be obtained from a feed including water and IPA by consumption of a minimum amount of energy.

### DESCRIPTION OF DRAWINGS

FIG. 1 shows a process of the above-described method;

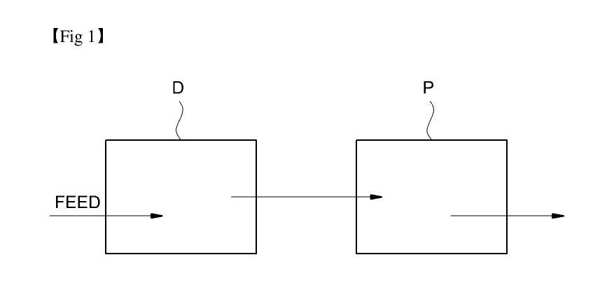

FIG. 2 shows a purification means used in the method;

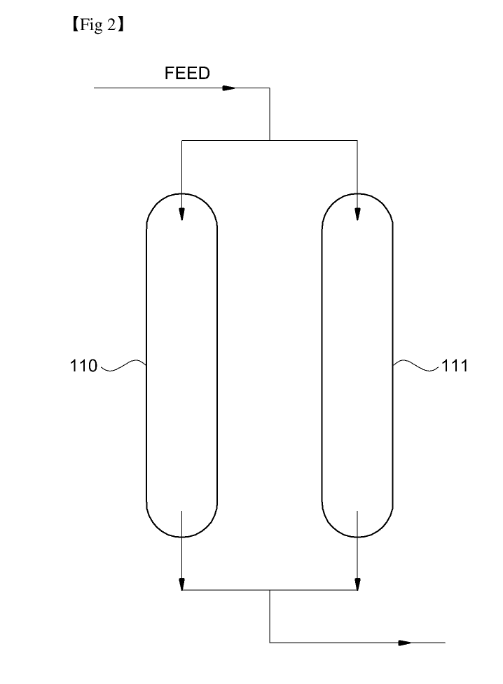

FIG. 3 shows a purification means used in the present method;

FIG. 4 shows a purification device according to a first example of the present invention; and

FIGS. 5 and 6 show a purification device according to a comparative example of the present invention.

### MODE FOR INVENTION

Hereinafter, the present invention will be described in further detail with reference to examples and comparative example, but the scopes of the method and device are not limited to the following examples.

### EXAMPLE 1

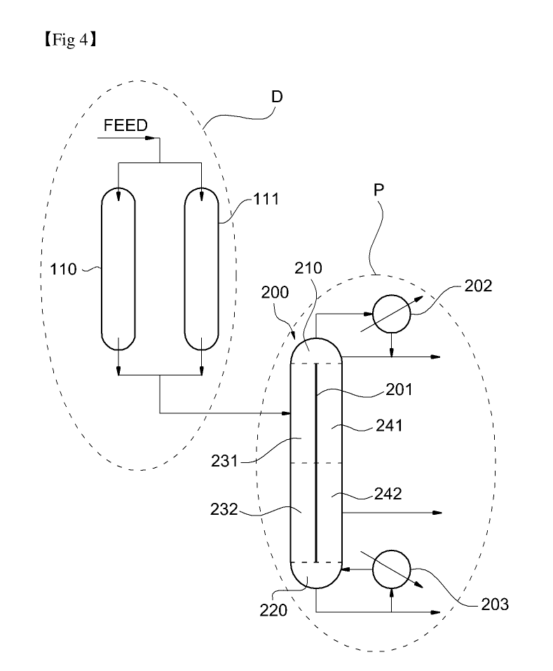

Isopropyl alcohol (IPA) was purified using a dehydration means and a divided wall column (DWC) connected with the dehydration means as shown in FIG. 4. Particularly, as a column charged with a molecular sieve, zeolite 3A having an effective average micropore size of approximately 3 Å, and two columns having a charged volume of approximately 3 m³ were used. Here, regeneration of the molecular sieve was performed using a means capable of providing nitrogen gas at approximately 230° C. and a flow rate of approximately 1,314 Nm³/hr. As the feed, a liquid feed including 98.6 wt % of IPA, approximately 3,200 ppm of water and approximately 1.08 wt % of other impurities was used. The feed was provided to the dehydration means at 90° C. and a dehydration process was performed such that the water content in the feed was approximately 300 ppm. Afterward, purification was performed by introducing the feed having a water content of approximately 300 ppm after the dehydration process into a feed inflow region of the DWC, specifically, 20 plates of the DWC having the number of theoretical plates of 90 plates, and a product including IPA was obtained from 60 plates of the DWC having the number of theoretical plates of 90 plates.

Here, the reflux ratio of a top region of the DWC was adjusted to 80, and operating temperature and pressure of the top region were adjusted to approximately 58° C. and 1.2 kg/cm², respectively. In this case, the operating temperature and pressure of a lower product outflow region were approximately 99° C. and 1.30 kg/cm², respectively, and operating temperature and pressure of the bottom region were approximately 117° C. and 1.37 kg/cm², respectively.

In this case, a content of a high boiling point component in IPA obtained from the lower product outflow region was detected at approximately 42 ppm.

### EXAMPLE 2

Purification was performed by the same method as described in Example 1, except that a reflux ratio of the top region was adjusted to 85.

### EXAMPLE 3

Purification was performed by the same method as described in Example 1, except that a reflux ratio of the top region was adjusted to 76.

### EXAMPLE 4

Purification was performed by the same method as described in Example 1, except that a product including IPA was obtained from 40 plates of the DWC having the number of theoretical plates of 90 plates.

### EXAMPLE 5

A process was performed by the same method as described in Example 1, except that a water content in a feed introduced into a purification means after passing through a dehydration means was approximately 500 ppm.

### EXAMPLE 6

Purification was performed by the same method as described in Example 1, except that a product including IPA was obtained from 70 plates of a DWC having the number of theoretical plates of 90 plates.

In this case, a content of a high-boiling-point component in the IPA obtained from a lower product outflow region was measured at approximately 52 ppm.

### EXAMPLE 7

Purification was performed by the same method as described in Example 1, except that operating temperature and pressure of a top region were adjusted to approximately 50° C. and 0.68 kg/cm², respectively.

In this case, operating temperature and pressure of a lower product outflow region were approximately 77.3° C. and 0.843 kg/cm², respectively, and operating temperature and pressure of the bottom region were approximately 104° C. and 0.91 kg/cm², respectively.

### EXAMPLE 8

Purification was performed by the same method as described in Example 1, except that operating temperature and pressure of a top region were adjusted to approximately 100° C. and 3.7 kg/cm², respectively.

In this case, operating temperature and pressure of a lower product outflow region were approximately 120° C. and 3.86 kg/cm², respectively, and operating temperature and pressure of the bottom region were approximately 156° C. and 3.93 kg/cm², respectively.

### COMPARATIVE EXAMPLE 1

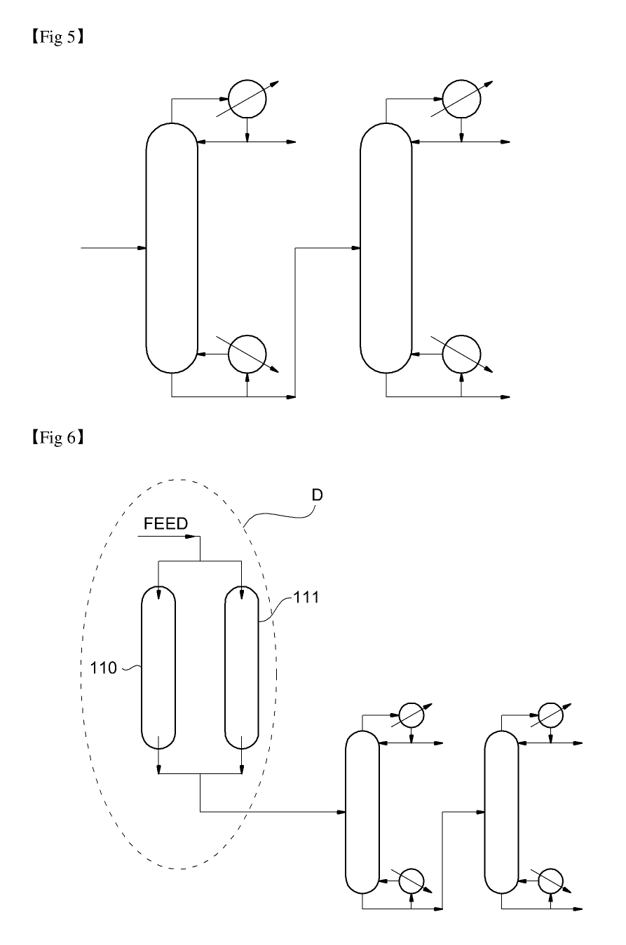

A liquid feed including 98.6 wt % of IPA, approximately 3,200 ppm of water and approximately 1.08 wt % of other impurities was purified by being introduced into a purification device in which two general columns were connected without passing through a dehydration process as shown in FIG. 5. In this case, top operating temperature and pressure of a first column were adjusted to approximately 76° C. and 1.12 kg/cm², respectively, and bottom operating temperature and pressure of the first column were adjusted to approximately 93° C. and 1.54 kg/cm², respectively. In addition, top operating temperature and pressure of a second column were adjusted to approximately 83° C. and 1.04 kg/cm², respectively, and bottom operating temperature and pressure of the second column were adjusted to approximately 110° C. and 1.18 kg/cm², respectively.

### COMPARATIVE EXAMPLE 2

As shown in FIG. 6, a process was performed by the same method as described in Example 1, except that a feed passing through a column charged with a molecular sieve was purified by being introduced into a purification device in which two general columns were connected, instead of a DWC. In this case, top operating temperature and pressure of a first column were adjusted to approximately 63° C. and 1.12 kg/cm², respectively, and bottom operating temperature and pressure of the first column were adjusted to approximately 93° C. and 1.54 kg/cm², respectively. In addition, top operating temperature and pressure of a second column were adjusted to approximately 83° C. and 1.04 kg/cm², respectively, and bottom operating temperature and pressure of the second column were adjusted to approximately 110° C. and 1.18 kg/cm², respectively.

### COMPARATIVE EXAMPLE 3

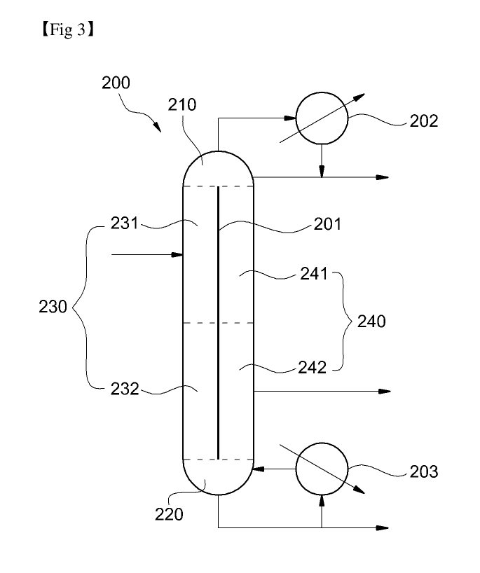

A process was performed by the same method as described in Example 1, except that a liquid feed including 98.6 wt % of IPA, approximately 3,200 ppm of water and approximately 1.08 wt % of other impurities was directly introduced into a DWC shown in FIG. 3 without going through a dehydration process. In this case, a reflux ratio of a top region of the DWC was adjusted to 52, top operating temperature and pressure of the DWC were adjusted to approximately 76° C. and 1.12 kg/cm², respectively, and bottom operating temperature and pressure of the DWC were adjusted to approximately 111° C. and 1.37 kg/cm², respectively.

### COMPARATIVE EXAMPLE 4

Purification was performed by the same method as described in Example 1, except that a product including IPA was obtained from 35 plates of a DWC having the number of theoretical plates of 90 plates.

### COMPARATIVE EXAMPLE 5

Purification was performed by the same method as described in Example 1, except that a product including IPA was obtained from 85 plates of a DWC having the number of theoretical plates of 90 plates.

In this case, a content of a high boiling point component in IPA obtained from a lower product outflow region was detected at approximately 590 ppm.

### COMPARATIVE EXAMPLE 6

Purification was performed by the same method as described in Example 1, except that a water content in a feed introduced into a purification means after a dehydration means was adjusted to approximately 700 ppm.

A total amount of energy and a water content in IPA used in Examples and Comparative Examples are summarized and listed in Tables 1 and 2.

TABLE 1

| | | Example 1 | Example 2 | Example 3 | Example 4 | Example 5 | Example 6 | Example 7 | Example 8 |
|---|---|---|---|---|---|---|---|---|---|
| Heat duty | Condenser | 1.49 | 1.6 | 1.43 | 1.49 | 1.78 | 1.49 | 1.43 | 1.59 |
| (Gcal/hr) | Reboiler | 1.47 | 1.58 | 1.41 | 1.47 | 1.76 | 1.47 | 1.37 | 1.74 |

| | | | | | | | | |
|---|---|---|---|---|---|---|---|---|
| Saved amount of | 1.55 | 1.44 | 1.61 | 1.55 | 1.26 | 1.55 | 1.65 | 1.28 |
| energy (Gcal/hr) | | | | | | | | |
| Energy saving rate (%) | 51% | 48% | 53% | 51% | 42% | 51% | 55% | 42% |
| Water content in IPA | 89 | 100 | 110 | 110 | 100 | 100 | 89 | 100 |
| (ppm) | | | | | | | | |

Saved amount of energy: Saved amount of energy compared to C. Example 1,
Energy saving rate: Energy saving rate compared to C. Example 1
* C. Example: Comparative Example

TABLE 2

| | | C. | C. | C. | C. | C. | C. |
|---|---|---|---|---|---|---|---|
| | | Example 1 | Example 2 | Example 3 | Example 4 | Example 5 | Example 6 |
| Heat duty | Condenser | 3.13 | 2.5 | 2.02 | 1.49 | 1.49 | 3.10 |
| (Gcal/hr) | Reboiler | 3.02 | 2.4 | 2 | 1.47 | 1.47 | 2.99 |

| | | | | | | |
|---|---|---|---|---|---|---|
| Saved amount of energy | 0 | 0.62 | 1.02 | 1.55 | 1.55 | 0.03 |
| (Gcal/hr) | | | | | | |
| Energy saving rate (%) | 0% | 21% | 34% | 51% | 51% | 1% |
| Energy saving rate (ppm) | 100 | 100 | 100 | 130 | 100 | 100 |

Saved amount of energy: Saved amount of energy compared to C. Example 1,
Energy saving rate: Energy saving rate compared to C. Example 1
* C. Example: Comparative Example

1. A method for purifying isopropyl alcohol, comprising:
removing water by providing a feed including isopropyl alcohol and water to a column charged with an adsorbent; and
performing purification by providing the feed having a water content adjusted by removing water from the column to a divided wall column.

2. The method according to claim 1, wherein the adsorbent includes a molecular sieve, a silica gel, activated alumina, activated carbon or an ion exchange resin.

3. The method according to claim 2, further comprising regenerating a molecular sieve in which dehydration is performed using a nitrogen gas.

4. The method according to claim 3, wherein the regeneration is performed at 175 to 320° C.

5. The method according to claim 1, wherein the removal of water includes providing a feed having a water content of 1,200 to 5,000 ppm to a column charged with an adsorbent, and adjusting a water content of the feed to 500 ppm or less in the column.

6. The method according to claim 1, wherein the performing of purification includes providing the feed in which a water content is adjusted to 500 ppm or less to the divided wall column by removing water from the column and, adjusting a water content of the feed to 150 ppm or less.

7. The method according to claim 1, wherein the divided wall column is divided into a feed inflow region, a top region, a bottom region and a product outflow region, and the product outflow region is divided into an upper product outflow region and a lower product outflow region, and
performing the purification includes providing the feed in which a water content is adjusted to 500 ppm or less by removing water from the column to the feed inflow region of the divided wall column, performing purification in the divided wall column, and obtaining a discharged product including isopropyl alcohol and having a water content of 150 ppm or less from a lower product outflow region of the divided wall column.

8. The method according to claim 7, wherein the discharged product including purified isopropyl alcohol and having a water content of 150 ppm or less is obtained from 50 to 90% of plates of the number of theoretical plates calculated based on a top of the divided wall column.

9. The method according to claim 7, wherein a temperature of the top region of the divided wall column is adjusted to 40 to 120° C.

10. The method according to claim 7, wherein a pressure of the top region of the divided wall column is adjusted to 0.1 to 10.0 kg/cm².

11. The method according to claim 9, wherein a temperature of a flow discharged from the lower product region of the divided wall column is 60 to 130° C.

12. The method according to claim 10, wherein a pressure of the lower product outflow region of the divided wall column is 0.3 to 6.0 kg/cm².

13. The method according to claim 9, wherein a temperature of the bottom region of the divided wall column is 80 to 160° C.

14. The method according to claim 10, wherein a pressure of the bottom region of the divided wall column is 0.3 to 6.0 kg/cm².

15. A device for purifying isopropyl alcohol, comprising:
a column charged with an adsorbent into which a feed including isopropyl alcohol and water is introduced, and discharging the feed by adjusting a water content of the feed; and
a divided wall column including a divided wall column into which the feed passing

16. The device according to claim 15, wherein the adsorbent includes a molecular sieve, a silica gel, activated alumina, activated carbon, or an ion exchange resin.

17. The device according to claim 16, wherein the molecular sieve includes a zeolite, silica-alumina or silicate alumina.

18. The device according to claim 16, wherein the molecular sieve has an average micropore size of 1.0 to 5.0 Å.

19. The device according to claim 16, wherein the molecular sieve has a specific surface area of 100 to 1,500 m³/g.

20. The device according to claim 15, wherein the divided wall column is divided into a feed inflow region, a top region, a bottom region and a product outflow region, and the product outflow region is divided into an upper product outflow region and a lower product outflow region, and
wherein the feed in which a water content is adjusted to 500 ppm or less by removing water from the column is provided to the feed inflow region of the divided wall column, and a discharged product including purified isopropyl alcohol and having a water content of 150 ppm or less is discharged from the lower product outflow region of the divided wall column.
